(19) United States
(12) Patent Application Publication
(10) Pub. No.: US 2015/0148706 A1
(43) Pub. Date: May 28, 2015

(54) MEDICAL DEVICES FOR ACCESSING BODY LUMENS

(71) Applicant: Boston Scientific Scimed, Inc., Maple Grove, MN (US)

(72) Inventor: William Abner, Marlborough, MA (US)

(21) Appl. No.: 14/549,191

(22) Filed: Nov. 20, 2014

Related U.S. Application Data
(60) Provisional application No. 61/908,843, filed on Nov. 26, 2013.

(51) Int. Cl. A61M 25/09

(52) U.S. Cl. CPC A61M 25/09; A61M 2025/09075; A61M 2025/09175; A61M 2025/09133
USPC 600/585

(57) ABSTRACT

Medical devices and methods for making and using medical devices are disclosed. An example medical device may include a medical guidewire including a core wire. The core wire may include a distal constant diameter, a first tapered section, an intermediate constant diameter section, a second tapered section, and a proximal constant diameter section. The distal end of the first tapered section may be attached to the proximal end of the distal constant diameter section such that a first inflection point is defined where the distal end of the first tapered section and the proximal end of the distal constant diameter section meet. The core wire may be configured such that when a first predetermined longitudinal force is applied to the distal end of the distal constant diameter section along the longitudinal axis, the distal constant diameter section prolapses such that a first loop is defined about the first inflection point.

### CROSS-REFERENCE TO RELATED APPLICATIONS

This application claims priority under 35 U.S.C. §119 to U.S. Provisional Application Ser. No. 61/908,843, filed Nov. 26, 2013, the entirety of which is incorporated herein by reference.

### TECHNICAL FIELD

The present disclosure pertains to medical devices, and methods for manufacturing medical devices. More particularly, the present disclosure pertains to medical device for accessing body lumens.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device includes a medical guidewire. The guidewire may include a core wire having a longitudinal axis. The core wire may include a distal constant diameter, a first tapered section, an intermediate constant diameter section, a second tapered section, and a proximal constant diameter section. The distal end of the distal constant diameter section may define a distal end of the core wire. The distal end of the first tapered section may be attached to the proximal end of the distal constant diameter section such that a first inflection point is defined where the distal end of the first tapered section and the proximal end of the distal constant diameter section meet. The distal end of the intermediate constant diameter section may be attached to the proximal end of the first tapered section. The distal end of the second tapered section may be attached to the proximal end of the intermediate constant diameter section such that a second inflection point is defined where the distal end of the second tapered section and the proximal end of the intermediate constant diameter section meet. The distal end of the proximal constant diameter section may be attached to the proximal end of the second tapered section. The core wire may be configured such that when a first predetermined longitudinal force is applied to the distal end of the distal constant diameter section along the longitudinal axis, the distal constant diameter section prolapses such that a first loop is defined about the first inflection point.

Another example medical guidewire may include a core wire comprising a superelastic material and having a longitudinal axis. The core wire may include a distal constant diameter section including a proximal end and a distal end. The distal end may define a distal end of the core wire. The distal constant diameter section may have a length in the range of 0.1 cm to 2.5 cm and a diameter in the range of 0.001 inches to 0.008 inches. The core wire may also include a tapered section having a proximal end and a distal end. The distal end may be attached to the proximal end of the distal constant diameter section such that a first inflection point is defined where the distal end of the tapered section and the proximal end of the distal constant diameter section meet. The tapered section may have a length in the range of 0.5 cm to 3 cm. The core wire may also include a proximal portion having a distal end attached to the proximal end of the tapered section. The core wire may be configured such that when a predetermined longitudinal force is applied to the distal end of the distal constant diameter section along the longitudinal axis, the distal constant diameter section prolapses such that a loop is defined about the inflection point.

Methods for gaining access to an opening in a body lumen are also disclosed. The methods may include providing or otherwise using a guidewire such as the guidewires disclosed herein. The method may also include contacting a distal end of the guidewire with tissue in or adjacent to the opening, applying a first predetermined longitudinal force to the distal end of the core wire along the longitudinal axis such that a loop is formed in the guidewire, and advancing the loop formed in the guidewire into the opening.

Example medical devices for accessing an opening to a body lumen are also disclosed. An example medical device may include an elongate shaft having a distal constant diameter section, a first tapered section attached to the distal constant diameter section, an intermediate constant diameter section attached to the first tapered section, a second tapered section attached to the intermediate constant diameter section, and a proximal constant diameter section attached to the second tapered section. The distal constant diameter section may have a length in the range of 0.1 cm to 2.5 cm. A first inflection point may be defined in the shaft where the distal constant diameter section and the first tapered section meet. The shaft may be configured to form a first loop at the first inflection point when subjected to a first pre-determined longitudinal force. A second inflection point may be defined in the shaft where the intermediate constant diameter section and the second tapered section meet. The shaft may be configured to form a second loop at the second inflection point when subjected to a second pre-determined longitudinal force. The first predetermined force may be in the range of 20 g to 200 g. The second predetermined force may be in the range of 250 g to 700.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional side view of an example guidewire;

FIG. 2 is a side view of an example core wire;

FIG. 3 is a side view of an example core wire with a loop formed therein at a first deflection point;

FIG. 4 is a side view of an example core wire with a loop formed therein at a second deflection point;

FIGS. 5-7 are plan views depicting an example guidewire being advanced to a target region.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

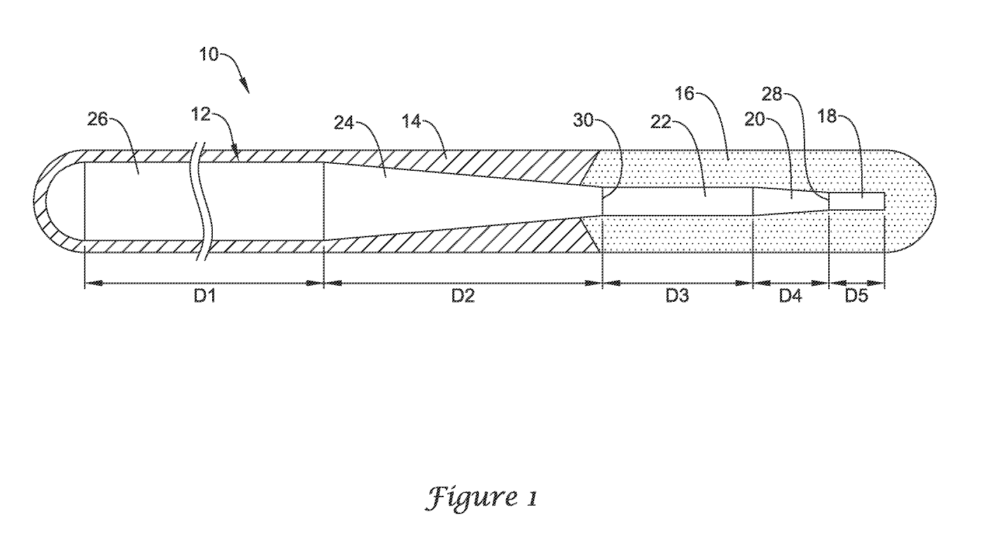

FIG. 1 is a partial cross-sectional side view of an example guidewire 10. Guidewire 10 may include a core wire 12. A sheath 14 may be disposed along at least a portion of core wire 12. In some embodiments, a tip member 16 may be disposed about a portion of core wire 12.

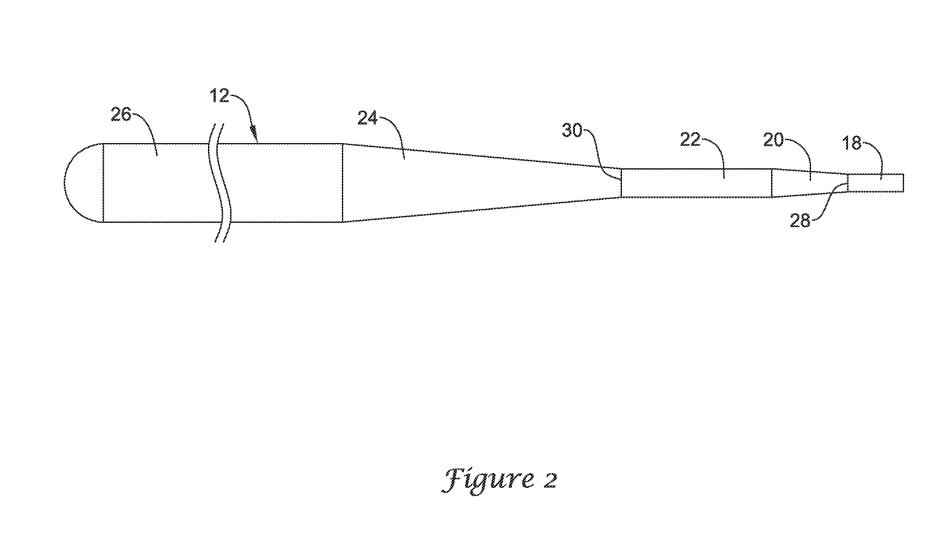

Core wire 12, which can also be seen in FIG. 2, may include a first or distal constant diameter section 18. A first tapered section 20 may be coupled to distal constant diameter section 18. For example, a proximal end of distal constant diameter section 18 may be attached to a distal end of first tapered section 20. A second or intermediate constant diameter section 22 may be coupled to first tapered section 20. For example, a proximal end of first tapered section 20 may be attached to a distal end of intermediate constant diameter section 22. A second tapered section 24 may be coupled to intermediate constant diameter section 22. For example, a proximal end of intermediate constant diameter section 22 may be attached to a distal end of second tapered section 24. A third or proximal constant diameter section 26 may be coupled to second tapered section 24. For example, a proximal end of second tapered section 24 may be attached to a distal end of proximal constant diameter section 26. These are just examples. Core wire 12 may include other sections.

In some embodiments, core wire 12 may be a unitary structure or otherwise formed from a single monolith of material. In other embodiments, core wire 12 may be formed from a plurality of different structures that are secured together. This may include one or more of sections 18/20/22/24/26 being separate structures that are secured to the remaining sections of core wire 12. In such embodiments, the one or more separate structures may be secured to remaining sections of core wire 12 using a suitable bonding technique such as welding, brazing, thermal bonding, adhesive bonding, mechanical bonding and/or the use of a mechanical connector, or the like.

The dimensions of core wire 12 may vary. Disclosed herein are some example dimensions for the various sections of core wire 12. These dimensions are meant to be examples and are not intended to be limiting. Other dimensions are contemplated.

In at least some embodiments, proximal constant diameter section 26 may have a length (represented in FIG. 1 as dimension D1) of about 200-600 cm (78.4-236.2 inches), or about 260-500 cm (102.4-196.9 inches). The diameter of proximal constant diameter section 26 may be about 0.01-0.04 inches, or about 0.015-0.030 inches, or about 0.023 inches.

Second tapered section 24 may have a length (represented in FIG. 1 as dimension D2) of about 1-50 cm (0.4-19.7 inches), or about 2-50 cm (0.8-19.7 inches), or about 2-15 cm (0.8-5.9 inches), or about 2-10 cm (0.8-3.9 inches), or about 5 cm (2 inches). The diameter of second tapered section 24 may vary from a diameter that is equal to or approximately equal to the diameter of proximal constant diameter section 26 to a diameter that is equal to or approximately equal to the diameter of intermediate constant diameter section 22. The transition in diameter may be a linear taper or non-linear taper. For example, the tapering of second tapered section 24 may be a parabolic taper, a curvilinear taper, a straight taper, a function of a non-linear equation (e.g., a second order equation, a third order equation, a fourth order equation, or the like), or the like. The taper may be constant along the length of second tapered section 24 or the taper may vary along the length. In some embodiments, the taper may include one or more steps in diameter.

Intermediate constant diameter section 22 may have a length (represented in FIG. 1 as dimension D3) of about 0.5-20 cm (0.2-7.9 inches), or about 1-10 cm (0.4-4 inches), or about 2.5-7.5 cm (1-3 inches). The diameter of intermediate constant diameter section 22 may be about 0.001-0.04 inches, or about 0.005-0.015 inches, or about 0.01 inches.

First tapered section 20 may have a length (represented in FIG. 1 as dimension D4) of about 0.1-10 cm (0.04-4 inches), or about 0.5-10 cm (0.2-4 inches), or about 0.5-3 cm (0.2-1.2 inches), or about 1 cm (0.4 inches). The diameter of first tapered section 20 may vary from a diameter that is equal to or approximately equal to the diameter of intermediate constant diameter section 22 to a diameter that is equal to or approximately equal to the diameter of distal constant diameter section 18. The transition in diameter may be a linear taper or non-linear taper. For example, the tapering of first tapered section 20 may be a parabolic taper, a curvilinear taper, a straight taper, a function of a non-linear equation (e.g., a second order equation, a third order equation, a fourth order equation, or the like), or the like. The taper may be constant along the length of first tapered section 20 or the taper may vary along the length. In some embodiments, the taper may include one or more steps in diameter.

Distal constant diameter section 18 may have a length (represented in FIG. 1 as dimension D5) of about 0.1-5 cm (0.04-2 inches), or about 0.1-2.5 cm (0.04-1 inches), or about 1.5 cm (0.6 inches). The diameter of distal constant diameter section 18 may be about 0.001-0.02 inches, or about 0.001-0.008 inches, or about 0.005 inches. In some embodiments, the ratio of length of distal constant diameter section 18 to the diameter of distal constant diameter section 18 may be in the range of about 100:1-1500:1, or about 200:1-1200:1, or about 800:1-1200:1.

In some embodiments, distal constant diameter section 18 may have a cross-sectional shape that is substantially round. In other embodiments, distal constant diameter section 18 may be flattened or otherwise have a non-circular cross-sectional shape.

Forming core wire 12 may include a grinding technique such as through the use of a centerless grinder. In some embodiments, a standard centerless grinder may be used to form core wire 12. In other embodiments, the centerless grinding teachnique may be modified to use a thinner cutting wheel, which may allow for greater precision and/or for the creature of increasing and decreasing tapers along the length of core wire 12. In some of these and in other embodiments, different grinding modes may be used to form the desired configuration of core wire 12 such as “OD” mode.

Guidewires for using in coronary interventions are often designed to have a relatively stiff proximal section for providing “pushability” and a relatively flexible distal region and/or tip. In addition, coronary guidewires may be designed to avoid kinking or prolapsing when being advanced through a blood vessel. A kinked or prolapsed guidewire may not be fully functional and may not be suitable for guiding other diagnostic and/or therapeutic devices to a target region.

Guidewire 10 may find utility for use in endoscope interventions such as accessing the common bile duct (and/or the biliary tree), the pancreatic duct (and/or the pancreatic tree), or the like. Unlike coronary guidewires, guidewire 10 is designed to have one or more pre-determined inflection points where guidewire 10 may prolapse or otherwise form a loop. This design may desirably allow guidewire 10 to be used in endoscopic interventions or otherwise be used along the biliary or pancreatic tree. For example, the ability of guidewire 10 to form a predictable, pre-determined loop configuration may desirably impact the ability of guidewire 10 to cannulate anatomical structures such as the papilla of Vater, gain access to body lumens such as the common bile duct and/or the pancreatic duct, or otherwise perform endoscopic interventions.

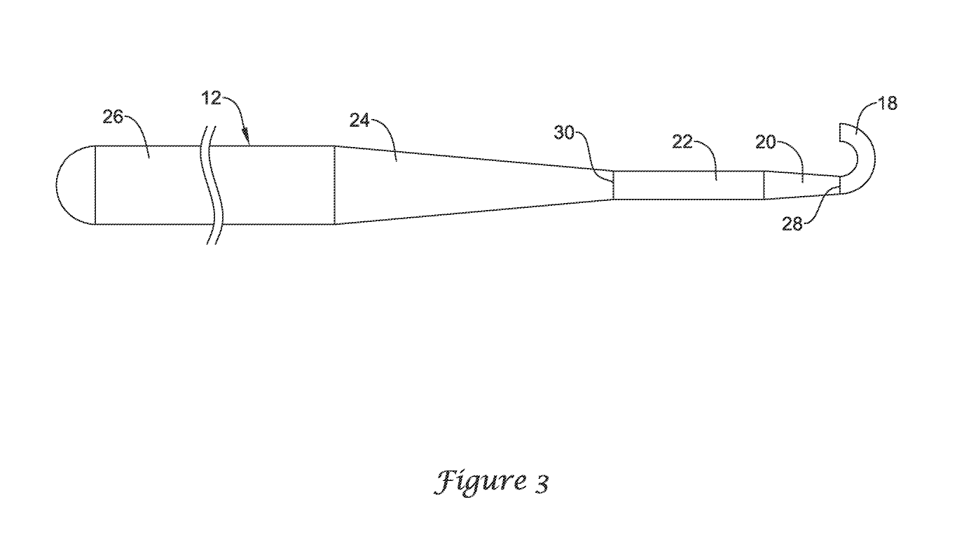

The design of core wire 12 may aid in guidewire 10 being able to form a predictable, pre-determined loop configuration. For example, a first inflection point 28 may be defined where distal constant diameter section 18 and first tapered section 20 meet. Because of the length of distal constant diameter section 18, first inflection point 28 may be positioned relatively close to the distal end of guidewire 10. When guidewire 10 is subjected to a suitable longitudinal force, distal constant diameter section 18 may prolapse or otherwise form a loop as shown in FIG. 3. In at least some embodiments, the amount of force may be relatively low. For example, guidewire 10 may form the loop at first inflection point 28 when subjected to about 20-200 g of longitudinal force, or about 20-200 g of longitudinal force. In some embodiments, the amount of longitudinal force that may cause guidewire 10 to form a loop at first inflection point 28 may be about 1.1-2.0 times the insertion force (e.g., the force needed to insert guidewire 10 into a catheter, endoscope, or another device), or about 1.2-1.5 times the insertion force, or about 1.25 times the insertion force.

The ability to form a relatively tight loop with a relatively small amount of force may be desirable. For example, cannulation methods for accessing the bile duct may be challenging. By quickly forming a tight loop, a clinician may be able to more efficiently navigate anatomical structures such as the papilla of Vater. Furthermore, because first inflection point 28 is positioned close to the distal end of guidewire 10, the loop formed when guidewire 10 may be shorter, more tightly associated with the rest of the guidewire (e.g., the radius of curvature may be kept to a minimum), and less force may be applied to the surrounding anatomy. Accordingly, guidewire 10 may form a loop more easily, may have a better “feel” for clinicians, and may also be able to revert back to a linear state more easily and/or with less trauma to the surrounding tissue. In addition, the guidewire 10 may be able to “flick” or otherwise snap back to a more “unlooped” state so that guidewire 10 can continue to advance through the anatomy.

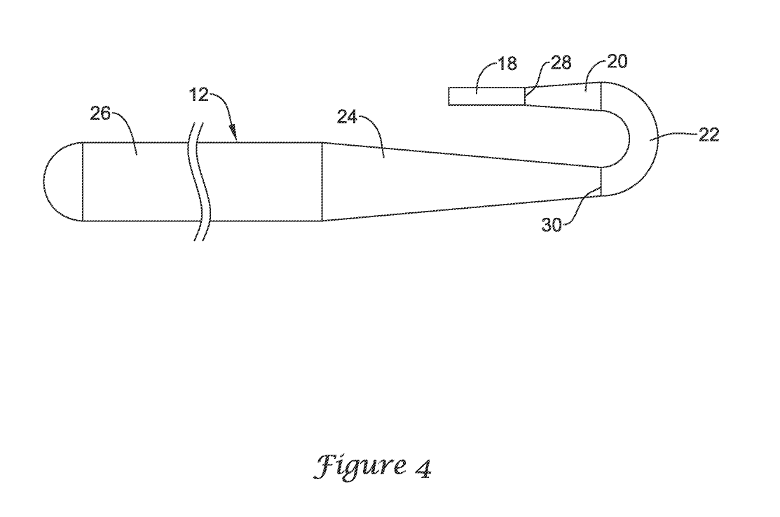

In at least some embodiments, guidewire 10 may include a plurality of inflection points. For example, a second inflection point 30 may be defined where intermediate constant diameter section 22 and second tapered section 24 meet. When guidewire 10 is subjected to a suitable longitudinal force, the sections of core wire 12 distal of second inflection point 30 may prolapse or otherwise form a loop as shown in FIG. 4. In at least some embodiments, the amount of force may greater than that of first inflection point 28. For example, guidewire 10 may form the loop at second inflection point 30 when subjected to about 250-700 g of longitudinal force. In some embodiments, the amount of longitudinal force that may cause guidewire 10 to form a loop at second inflection point 30 may be about 1.75-2.5 times the insertion force (e.g., the force needed to insert guidewire 10 into a catheter, endoscope, or another device), or about 1.8-2.2 times the insertion force, or about 2 times the insertion force.

By having inflection points 28/30, guidewire 10 may form a loop at a specific location along the length thereof, with a specific force, and with a controlled (and/or reduced) circumferential force on the body lumen. In at least some embodiments, inflection points 28/30 may be defined where a constant diameter section meets a tapered section. However, other arrangements are contemplated. For example, other structural modifications may also be utilized to define an inflection point such as stiffened regions of core wire 12, structures secured to core wire 12 to impart stiffness, changes in the location and/or composition of the sections of core wire 12, changes in the composition and/or location of sheath 14 and/or tip member 16, or the like.

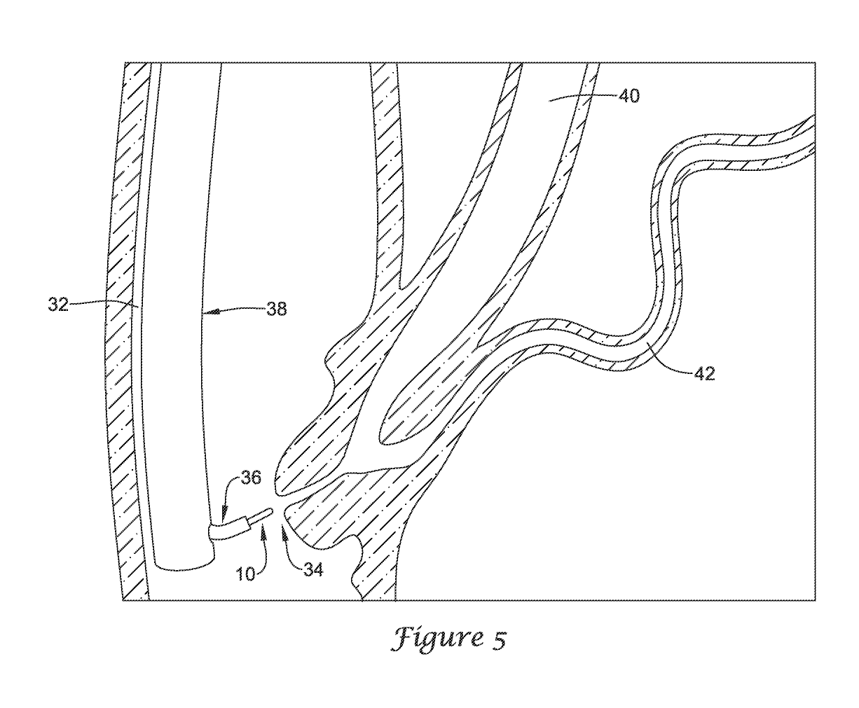

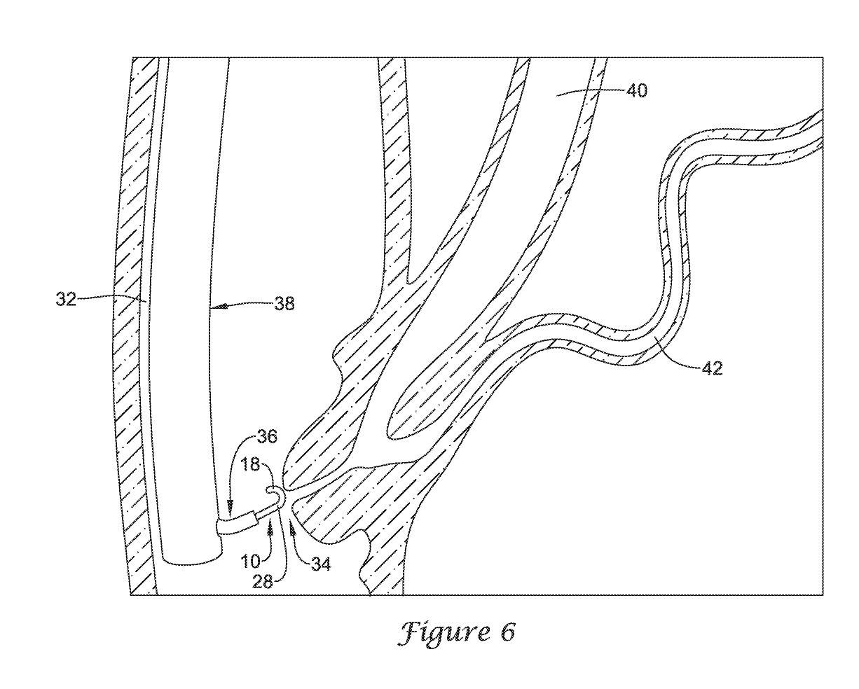

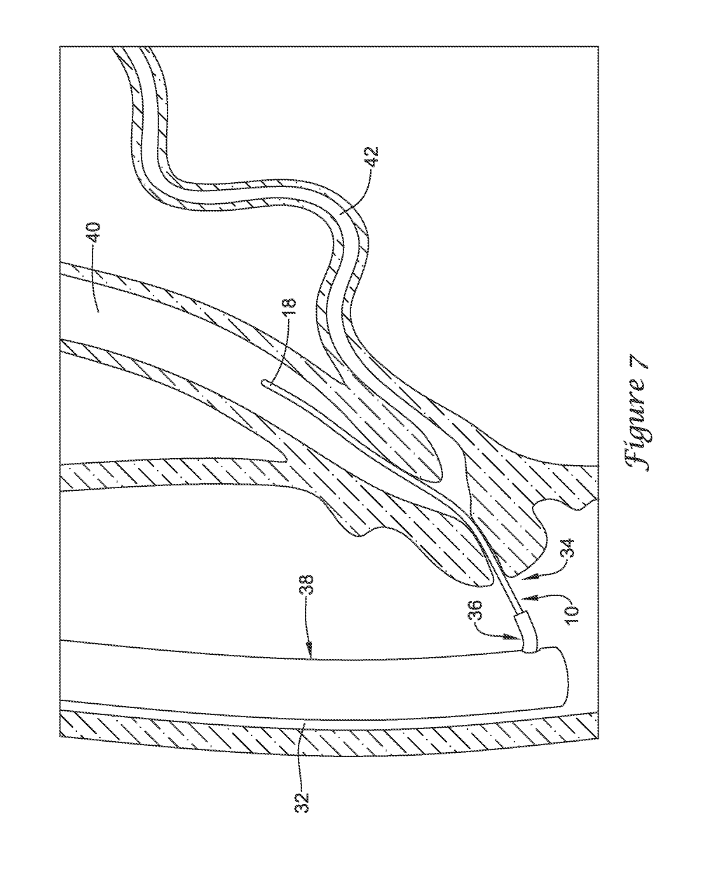

In use, guidewire 10 may be advanced through a body lumen such as the duodenum 32 to a position adjacent to the papilla of Vater 34 as shown in FIG. 5. This may include the use of a cannulation or guide catheter 36 and/or an endoscope 38. Guidewire 10 may be advanced out from guide catheter 36 and into engagement with the papilla of Vater 34. If a suitable amount of resistance is encountered, guidewire 10 may prolapse or form a loop a first inflection point 28 as shown in FIG. 6. Guidewire 10 may be advanced through the papilla of Vater 34 to a suitable diagnostic and/or treatment site such as along the biliary tract (e.g., the common bile duct 40) or the pancreatic tract (e.g., the pancreatic duct 42). For example, guidewire 10 is shown advanced into the common bile duct 40 in FIG. 7.

The materials that can be used for the various components of guidewire 10 may include metals, metal alloys, polymers, metal-polymer composites, ceramics, combinations thereof, and the like, or other suitable materials. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of core wire 12 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of guidewire 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into guidewire 10. For example, core wire 12, or portions thereof, may be made of a material that does not substantially distort the image and create substantial artifacts (i.e., gaps in the image). Certain ferromagnetic materials, for example, may not be suitable because they may create artifacts in an MRI image. Core wire 12, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Referring now to core wire 12, the entire core wire 12 can be made of the same material along its length, or in some embodiments, can include portions or sections made of different materials. In some embodiments, the material used to construct core wire 12 is chosen to impart varying flexibility and stiffness characteristics to different portions of core wire 12. In embodiments where different portions of core wire 12 are made of different materials, the different portions can be connected using a suitable connecting technique and/or with a connector. For example, the different portions of core wire 12 can be connected using welding (including laser welding), soldering, brazing, adhesive, or the like, or combinations thereof. These techniques can be utilized regardless of whether or not a connector is utilized.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical guidewire, comprising:
a core wire having a longitudinal axis, the core wire including:
a distal constant diameter section having a distal end and a proximal end, the distal end of the distal constant diameter section defining a distal end of the core wire;
a first tapered section having a distal end and a proximal end, the distal end of the first tapered section being attached to the proximal end of the distal constant diameter section such that a first inflection point is defined where the distal end of the first tapered section and the proximal end of the distal constant diameter section meet;
an intermediate constant diameter section having a proximal end and a distal end, the distal end of the intermediate constant diameter section being attached to the proximal end of the first tapered section;
a second tapered section having a proximal end and a distal end, the distal end of the second tapered section being attached to the proximal end of the intermediate constant diameter section such that a second inflection point is defined where the distal end of the second tapered section and the proximal end of the intermediate constant diameter section meet; and
a proximal constant diameter section having a proximal end and a distal end, the distal end of the proximal constant diameter section being attached to the proximal end of the second tapered section;
wherein the core wire is configured such that when a first predetermined longitudinal force is applied to the distal end of the distal constant diameter section along the longitudinal axis, the distal constant diameter section prolapses such that a first loop is defined about the first inflection point.

2. The guidewire of claim 1, wherein the core wire is configured such that when the first loop is defined about the first inflection point, the first loop defines a distal extremity of the core wire, and when a second predetermined longitudinal force is applied to the distal extremity along the longitudinal axis, the intermediate constant diameter section prolapses to define a second loop about the second inflection point.

3. The guidewire of claim 1, wherein the first predetermined force is less than the second predetermined force.

4. The guidewire of claim 1, wherein the first predetermined force is in the range of 20 g to 200 g.

5. The guidewire of claim 1, wherein the second predetermined force is in the range of 250 g to 700 g.

6. The guidewire of claim 1, wherein the distal constant diameter section has a length in the range of 0.1 cm to 2.5 cm.

7. The guidewire of claim 1, wherein the distal constant diameter section has a diameter in the range of 0.001 inches to 0.008 inches.

8. The guidewire of claim 1, wherein the first tapered section has a length in the range of 0.5 cm to 3 cm.

9. The guidewire of claim 1, wherein the intermediate constant diameter section has a length in the range of 1 cm to 10 cm.

10. The guidewire of claim 1, wherein the intermediate constant diameter section has a diameter in the range of 0.005 inches to 0.015 inches.

11. The guidewire of claim 1, wherein the second tapered section has a length in the range of 2 cm to 50 cm.

12. The guidewire of claim 1, wherein the proximal constant diameter section has a diameter in the range of 0.015 inches to 0.030 inches.

13. The guidewire of claim 1, wherein the ratio of the length of the distal constant diameter section to the diameter of the distal constant diameter section is in the range of 800:1 to 1200:1.

14. The guidewire of claim 1, wherein the core wire comprises a superelastic material.

15. The guidewire of claim 14, wherein the core wire comprises a superelastic nickel-titanium alloy.

16. The guidewire of claim 1, further comprising an outer sheath disposed along at least a portion of the core wire.

17. The guidewire of claim 1, further comprising a tip member disposed along at least the distal constant diameter section.

18. A medical guidewire, comprising:
a core wire comprising a superelastic material and having a longitudinal axis, the core wire including:
a distal constant diameter section including a proximal end and a distal end, the distal end defining a distal end of the core wire;
wherein the distal constant diameter section has a length in the range of 0.1 cm to 2.5 cm and a diameter in the range of 0.001 inches to 0.008 inches;
a tapered section having a proximal end and a distal end, the distal end being attached to the proximal end of the distal constant diameter section such that a first inflection point is defined where the distal end of the tapered section and the proximal end of the distal constant diameter section meet;
wherein the tapered section has a length in the range of 0.5 cm to 3 cm;
a proximal portion having a distal end attached to the proximal end of the tapered section;
wherein the core wire is configured such that when a predetermined longitudinal force is applied to the distal end of the distal constant diameter section along the longitudinal axis, the distal constant diameter section prolapses such that a loop is defined about the inflection point.

19. The guidewire of claim 18, wherein the predetermined force is in the range of 20 g to 200 g.

20. A medical device for accessing an opening to a body lumen, the medical device comprising:
an elongate shaft having a distal constant diameter section, a first tapered section attached to the distal constant diameter section, an intermediate constant diameter section attached to the first tapered section, a second tapered section attached to the intermediate constant diameter section, and a proximal constant diameter section attached to the second tapered section;
wherein the distal constant diameter section has a length in the range of 0.1 cm to 2.5 cm;
wherein a first inflection point is defined in the shaft where the distal constant diameter section and the first tapered section meet;
wherein the shaft is configured to form a first loop at the first inflection point when subjected to a first pre-determined longitudinal force;
wherein a second inflection point is defined in the shaft where the intermediate constant diameter section and the second tapered section meet;
wherein the shaft is configured to form a second loop at the second inflection point when subjected to a second pre-determined longitudinal force;
wherein the first predetermined force is in the range of 20 g to 200 g; and
wherein the second predetermined force is in the range of 250 g to 700 g.
